(19) United States
(12) Patent Application Publication
(10) Pub. No.: US 2026/0355232 A1
(43) Pub. Date: Nov. 12, 2026

(54) METHODS OF MANAGING GLYCEMIC CONTROL IN PRADER-WILLI SYNDROME PATIENTS

(71) Applicant: Soleno Therapeutics, Inc, Redwood City, CA (US)

(72) Inventor: Neil M. Cowen, Carlsbad, CA (US)

(21) Appl. No.: 19/748,844

(22) Filed: Jul. 21, 2026

Related U.S. Application Data
(63) Continuation of application No. 19/566,422, filed on Mar. 13, 2026.
Application No. 19/566,422 is a continuation of application No. PCT/IB2024/058904, filed on Sep. 13, 2024.
(60) Provisional application No. 63/583,179, filed on Sep. 15, 2023.

(51) Int. Cl. A61K 33/06; A61K 9/20; A61K 31/549; A61P 3/02; A61P 3/04; A61P 3/10

(52) U.S. Cl. CPC A61K 33/06; A61K 9/2009; A61K 9/2013; A61K 9/2031; A61K 9/2054; A61K 31/549; A61P 3/02; A61P 3/04; A61P 3/10

(57) ABSTRACT

Provided herein are methods of managing glycemic control in a subject with Prader-Willi Syndrome in need thereof, comprising co-administering daily to the subject with Prader-Willi Syndrome in need thereof, a daily dose of a K_{ATP} channel opener, or a pharmaceutically acceptable salt thereof, and a daily dose of a magnesium supplement, for at least 1 week.

### CROSS-REFERENCE TO RELATED APPLICATIONS

This application is a continuation of U.S. patent application Ser. No. 19/566,422, filed Mar. 13, 2026, which is a continuation of International Patent Application No. PCT/IB2024/058904, filed Sep. 13, 2024, which claims priority to U.S. Provisional Application No. 63/583,179, filed Sep. 15, 2023, each of which is herein incorporated by reference in its entirety for all purposes.

### BACKGROUND

Prader-Willi Syndrome (“PWS”) is a rare, complex genetic neurobehavioral/metabolic disorder with an estimated birth incidence of 1:15,000 to 1:20,000. PWS arises from lack of expression of paternally inherited imprinted genes on chromosome 15q11-q13 caused by a paternal deletion, maternal uniparental disomy 15, or an imprinting center defect, resulting in hypothalamic dysfunction. Clinical features of PWS include hypotonia and feeding difficulties in infancy and sustained accumulation of excess body fat beginning in early childhood. Hyperphagia, which occurs around an average age of 8-9 years in PWS patients, presents as food obsession, aggressive food seeking, and lack of satiety, with progression to severe obesity if energy intake is not restricted. PWS is also associated with intellectual disability, low muscle mass, neuroendocrine abnormalities, including growth hormone and gonadotropin deficiency, behavioral problems, including aggression, anxiety and compulsivity, and elevated incidence of early mortality.

It has been found that certain metabolic signs or symptoms of subjects with PWS and other syndromic obesities can be alleviated with K_{ATP} channel openers. For example, the K_{ATP} channel opener Diazoxide Choline formulated as an extended-release tablet formulation can be used to treat hyperphagia in subjects with Prader-Willi Syndrome, as described in the following: WO2016/077629, U.S. Pat. Nos. 9,757,384, 10,058,557, 10,456,408, 10,874,676, US 2021/0322434, each of which is incorporated by reference in its entirety herein. However, there is still a need for additional treatment regimens to address the numerous signs and symptoms of Prader-Willi Syndrome.

### SUMMARY

Disclosed herein is a method of managing glycemic control in a subject with Prader-Willi Syndrome in need, comprising co-administering daily to the subject with Prader-Willi Syndrome in need, a daily dose of a K_{ATP} channel opener, or a pharmaceutically acceptable salt thereof, and a daily dose of a magnesium supplement, for at least 1 week.

In some embodiments, the K_{ATP} channel opener, or pharmaceutically acceptable salt thereof, is diazoxide choline.

In some embodiments, the co-administration is daily for at least 4 weeks, for at least 8 weeks, for at least 13 weeks, or for at least 1 year.

In some embodiments, a total daily dose of the K_{ATP} channel opener is about 25 mg to about 580 mg, about 100 mg to about 600 mg, about 250 mg to about 500 mg, or a corresponding amount of a pharmaceutically acceptable salt thereof.

In some embodiments, the total daily dose of the K_{ATP} channel opener, or pharmaceutically acceptable salt thereof, is administered once daily. In some embodiments, the total daily dose of the K_{ATP} channel opener, or pharmaceutically acceptable salt thereof, is formulated as an extended-release formulation.

In some embodiments, the total daily dose of the magnesium supplement is administered once daily.

In some embodiments, the total daily dose of magnesium supplement and the daily dose of K_{ATP} channel opener, or pharmaceutically acceptable salt thereof, are each administered once daily. The total daily dose of K_{ATP} channel opener, or pharmaceutically acceptable salt thereof, may be administered once daily and the magnesium supplement is administered two or more times daily. In some embodiments, the total daily dose of each of the K_{ATP} channel opener, or pharmaceutically acceptable salt thereof, and/or the magnesium supplement, are administered as two or more dosage forms. In some embodiments, the magnesium supplement is a solid dosage form.

In some embodiments, the subject with Prader-Willi Syndrome in need thereof is at risk for hypomagnesemia.

In some embodiments, the subject with Prader-Willi Syndrome at risk for hypomagnesemia is about 17 years of age or older, is obese, has a Type 1 deletion within 15q11.2-q13 encompassing breakpoints BP1 to BP3 in Chromosome 15, has a serum magnesium concentration threshold of less than about 2.2 mg/dL prior to co-administration, and/or has a serum magnesium concentration of less than about 2.0 mg/dL prior to co-administration.

In some embodiments, the subject with Prader-Willi Syndrome's hemoglobin A1C (HbA1c) decreases by at least 0.5% compared to prior to co-administration, decreases by at least 0.7% compared to prior to co-administration, or decreases by at least 0.8% compared to prior to co-administration.

In some embodiments, the subject with Prader-Willi Syndrome's serum magnesium concentration after at least 1 week of administration is greater than about at least 1.7 mg/dL, greater than about at least 2.00 mg/dL, or greater than about at least 2.07 mg/dL.

Also disclosed herein is the use of a magnesium supplement in the manufacture of a medicament for managing glycemic control in a subject with Prader-Willi Syndrome (PWS) in need thereof. In some embodiments, the use of a magnesium supplement in the manufacture of the medicament for managing glycemic control may include managing glycemic control in a subject with PWS co-administered a daily dose of a K_{ATP} channel opener or a pharmaceutically acceptable salt thereof. Also disclosed herein is the use of a magnesium supplement characterized by being for the manufacture of a medicament for the management of glycemic control or characterized in that it is useful for the preparing a medicament for the management of glycemic control in a subject with PWS in need thereof. Also disclosed herein are magnesium supplements for use in the management of glycemic control in a subject with PWS in need thereof. Also disclosed herein are uses of magnesium supplements for the management of subjects of PWS in need of management of glycemic control.

Additional embodiments, features, and advantages of the invention will be apparent from the following detailed description and through practice of the invention.

### BRIEF DESCRIPTION OF DRAWINGS

FIG. 1 depicts a graph showing the relationship between changes in serum magnesium concentrations and HbA1c from baseline levels compared to changes in concentrations of serum magnesium and HbA1c after 1 year of administration of a K_{ATP} channel opener to a population of subjects with PWS.

FIG. 2 depicts a graph showing the relationship between changes in concentrations of serum magnesium and HbA1c from baseline levels compared to changes in concentrations of serum magnesium and HbA1c after 1 year of administration of a K_{ATP} channel opener to a population of subjects with PWS having a baseline serum magnesium concentration greater than 2.2 mg/dL.

FIG. 3 depicts a graph showing the relationship between changes in concentrations of serum magnesium and HbA1c from baseline levels compared to changes in concentrations of serum magnesium and HbA1c after 1 year of administration of a K_{ATP} channel opener to a population of subjects with PWS having a baseline serum magnesium concentration between 2.0 mg/dL and 2.2 mg/dL.

FIG. 4 depicts a graph showing the relationship between changes in concentrations of serum magnesium and HbA1c from baseline concentrations compared to changes in concentrations of serum magnesium concentrations and HbA1c after 1 year of administration of a K_{ATP} channel opener to a population of subjects with PWS having a baseline serum magnesium concentration less than 2.0 mg/dL (e.g., hypomagnesemia).

FIG. 5 depicts a graph showing the relationship between changes in concentrations of serum magnesium and HbA1c from baseline concentrations compared to changes in concentrations of serum magnesium and HbA1c after 1 year of administration of a K_{ATP} channel opener to a population of subjects with PWS having a Type 1 deletion and a baseline serum magnesium concentration less than 2.2 mg/dL.

FIG. 6 depicts a graph showing the relationship between changes in concentrations of serum magnesium and HbA1c from baseline concentrations compared to changes in concentrations of serum magnesium and HbA1c after 1 year of administration of a K_{ATP} channel opener to a population of subjects with PWS lacking a Type 1 deletion and having a baseline serum magnesium concentration less than 2.2 mg/dL.

FIG. 7 depicts a graph showing the relationship between changes in concentrations of serum magnesium and HbA1c from baseline concentrations compared to changes in serum magnesium and HbA1c after 1 year of administration of a K_{ATP} channel opener to a population of obese subjects with PWS having a baseline serum magnesium concentration less than 2.2 mg/dL.

### DETAILED DESCRIPTION

### Prader-Willi Syndrome

As previously noted, Prader-Willi Syndrome (“PWS”) is a rare, complex genetic neurobehavioral/metabolic disorder with an estimated birth incidence of 1:15,000 to 1:20,000. PWS arises from lack of expression of paternally inherited imprinted genes on chromosome 15q11-q13 caused by a paternal deletion, maternal uniparental disomy 15, or an imprinting center defect, resulting in hypothalamic dysfunction.

While Prader-Willi Syndrome is a genetic condition, it occurs de novo in nearly all cases. As such, individuals with PWS can be thought of as being sampled from the larger population without regard to ethnicity. It has been found that certain metabolic signs or symptoms of subjects with PWS and other syndromic obesities can be alleviated with K_{ATP} channel openers, such as diazoxide choline. Descriptions of the use of diazoxide in the treatment of PWS and Smith Magenis Syndrome (SMS) subjects can be found in, for example, in U.S. Pat. No. 9,757,384, 10,058,557, 10,456,408, 10,874,676, Patent Publication No: US 2021/0322434, Patent Publication No: US 2024/0207284, U.S. Pat. No. 9,782,416, Patent Publication No: US 2020/0237772, Patent Publication No: US 2020/0222417, or U.S. Pat. No. 10,085,998, each of which is incorporated by reference herein.

However, administration of a K_{ATP} channel opener in some subjects may result in decreases in serum magnesium levels. Decreased magnesium levels below a threshold (e.g., hypomagnesemia) can lead to adverse effects such as impaired glycemic control. It may be beneficial to subjects with PWS that have impaired or compromised glycemic control and are identified with hypomagnesemia to be able to maintain glycemic control more effectively through magnesium supplementation. Disclosed herein are methods of managing glycemic control in a Prader-Willi Syndrome subject in need thereof that address the foregoing through daily co-administration of a daily dose of a K_{ATP} channel opener, or a pharmaceutically acceptable salt thereof, and a daily dose of a magnesium supplement, for at least 1 week.

### K_{ATP} Channel Openers

K_{ATP} channel openers can help regulate metabolic disorders by increasing the probability that the K_{ATP} channel is open at a given time. A K_{ATP} channel is an ATP-sensitive potassium channel that is gated by ATP and ADP. These channels are located both on the plasma membrane and on the mitochondria inner membrane. K_{ATP} channels relate the cell membrane potential to the metabolic status of the cell.

The K_{ATP} channel is an octameric structure. The channel consists of 4 copies of the sulfonylurea receptor (consisting of SUR1, SUR2A or SUR2B) and 4 copies of Kir (Kir6.1 or Kir6.2), an inwardly rectifying potassium channel. The genes encoding the channel reside on chromosomes 11p15.1 (ABCC8 encoding SUR1, and KCNJ11 encoding Kir6.2) and 12p12.1 (ABCC9 encoding SUR2 and KCNJ8 encoding Kir6.1). The channel assembles in the endoplasmic reticulum and is exported to the cell membrane. Changes in the surface density of the channel affect typical roles of the channel including the regulation of insulin secretion.

K_{ATP} channels undergo multiple rounds of rapid constitutive endocytosis and subsequent recycling to the plasma membrane. The rate of recycling is fairly rapid, with an equivalent to around ~80% of cell surface channels undergoing recycling within 10-15 min. It is evident that both internalization and recycling of K_{ATP} channels occur on a rapid time scale.

K_{ATP} channel openers are compositions that can increase the probability of opening K_{ATP} channels. The methods herein disclose treatment of subjects with a K_{ATP} channel opener or a pharmaceutically acceptable salt thereof. Examples of K_{ATP} channel openers include: diazoxide, BPDZ62, BPDZ73, NN414 and BPDZ154, levcromakalim, Y-26763, P1075, ZA226600, ZD0947, A278637, VU0071063, QLS-101, AO25, AO44, BPDZ42, BPDZ44, BPDZ141, BPDZ168, BPDZ187, BPDZ211, BPDZ216, BPDZ259, BPDZ260, BPDZ265, BPDZ281, BPDZ311, BPDZ678, BPDZ711, SR47063, VU0071063, VU0500330, VU0634148, VU6050536, VU6050189, VU6049921, VU6050319, VU6050322, VU6048194, VU6048045, VU6049921, VU6051543, VU6051545, and VU6051546, and as identified in U.S. Pat. No. 9,782,416, which is incorporated by reference in its entirety herein.

One example of a KAIP channel opener is diazoxide. Diazoxide is a potent opener of the K_{ATP} channel and is capable of crossing the blood-brain barrier. Activating the K_{ATP} channel in neuropeptide Y(NPY)/Agouti Related-Protein (AgRP) neurons in the hypothalamus results in reduced secretion of NPY and AgRP, potent endogenous appetite stimulatory neuropeptides, potentially contributing to a reduction in hyperphagia. These actions of the drug are complemented by activating the K_{ATP} channel in the dorsal motor nucleus of vagus nerve, pancreatic B-cells, and adipocytes to reduce hyperinsulinemia and excess body fat, and improve insulin and leptin resistance, and satiety. These effects have been confirmed in animal models of hyperphagic obesity including a model of PWS of a Magel-2 null mouse (Cowen and Bhatnagar Genes 2020; 11:450. Bischof and Wevrick Mol. Genet. Metab. 2018; 123(4):511-517).

The increased solubility of diazoxide choline and its slow release from an extended-release once-per-day tablet formulation (e.g., also referred to as a diazoxide extended-release formulation, which may be diazoxide or a pharmaceutically acceptable salt thereof including an extended-release formulation of diazoxide choline, including those described in Example 1), facilitate the absorption of diazoxide throughout the gastrointestinal tract, resulting in stable circulating concentrations. Examples of diazoxide extended-release formulations are described in U.S. Pat. No. 9,782,416, or US 2020/0237772, each of which are incorporated by reference in their entirety herein. Various references describing the preparation of diazoxide choline and additional salts include U.S. Pat. Nos. 7,572,789, 9,381,202, 10,085,998, US 2020/0222417, each of which is incorporated by reference in its entirety herein.

Administration of K_{ATP} Channel Opener to a Subject with PWS

In some embodiments, diazoxide, or a pharmaceutically acceptable salt thereof, including diazoxide choline, may be administered to a subject with PWS to achieve therapeutic outcomes while reducing the incidence of adverse effects. In some embodiments, diazoxide, or a pharmaceutically acceptable salt thereof, including diazoxide choline, may be administered to the subject with PWS at a dosage including from about 10 mg to about 100 mg, about 100 mg to about 200 mg, about 200 mg to about 300 mg, about 300 mg to about 500 mg, about 500 mg to about 600 mg, including from about 25 mg to about 580 mg. In some embodiments, diazoxide may be administered to the subject with PWS at a dosage including from about 15 mg to about 400 mg, including about 17 mg to about 400 mg, about 17 mg to about 350 mg, about 17 mg to about 300 mg, about 17 mg to about 250 mg, about 17 mg to about 200 mg, about 17 mg to about 150 mg, about 17 mg to about 100 mg, or about 17 mg to about 50 mg. In some embodiments, diazoxide may be administered to the subject with PWS at a dosage from about 50 mg to about 400 mg including from about 100 mg to about 400 mg, about 150 mg to about 400 mg, about 200 mg to about 400 mg, about 250 mg to about 400 mg, or about 300 mg to about 400 mg.

As will be appreciated by the skilled artisan, administration of diazoxide, or a pharmaceutically acceptable salt thereof, to the subject with PWS may be varied according to frequency, mechanisms of delivery, or dosage forms based on the signs or symptoms being exhibited by the subject, the age of the subject, etc. and as guided by a skilled physician. For example, the diazoxide, or a pharmaceutically acceptable salt thereof, may be administered to the subject with PWS using the oral dosage form of a tablet or a capsule. The diazoxide or the pharmaceutically acceptable salt thereof may be administered to the subject with PWS once daily (e.g., every about 24 hours), twice daily (e.g., two times per every about 24 hours), thrice daily (e.g., three times per every about 24 hours) or the like. In some embodiments, the diazoxide or the pharmaceutically acceptable salt thereof may be administered to the subjects with PWS using various mechanisms of delivery including, but not limited to, oral delivery, sublingual delivery, intravenous delivery, or the like. In certain embodiments, the administration of diazoxide or the pharmaceutically acceptable salt thereof to the subjects with PWS, if via oral delivery, may include types of oral delivery, such as via a capsule or tablet.

In some embodiments, the dosage forms may include extended-release variations. For example, diazoxide extended-release formulations may therefore result in better tolerability and efficacy at lower doses. In some embodiments, an extended-release formulation may include pharmaceutical product that may provide both the delay of release of the active pharmaceutical upon administration (e.g., pH-sensitive excipients where release begins after entry into the intestines) and controlled release for sustained release over a period of time (e.g., utilizing excipients that slow release compared to immediate release formulations but where release begins shortly after ingesting the formulation). In some embodiments, the K_{ATP} channel opener may include an extended-release formulation of diazoxide, or a pharmaceutically acceptable salt thereof, including diazoxide choline. Exemplary extended-release diazoxide choline formulations are taught in Example 1. In some embodiments, the extended-release diazoxide choline formulation is a formulation such as described in Example 1 (e.g., Tables 1.0 and 1.1 formulations such as any one of formulations A-M). In some embodiments, the formulation is formulation A, formulation B, formulation C, formulation D, formulation E, formulation F, formulation G, formulation H, formulation I, formulation J, formulation K, formulation L, formulation M, or the like.

Other extended-release diazoxide or diazoxide choline formulations may be found in U.S. Pat. Nos. 9,782,416, 7,799,777, 7,572,789, 9,381,202, 10,085,998, 11,045,478, 11,786,536, US patent publication no. 2024/0252507, U.S. Pat. No. 9,757,384, U.S. Pat. Nos. 10,058,557, and 10,456,408, each of which is incorporated by reference in its entirety herein.

### Administration of K_{ATP} Channel Opener

In some embodiments, the K_{ATP} channel opener may be administered to the subject with PWS for any length of time greater than or equal to about 1 week. For example, the K_{ATP} channel opener may be administered to the subject for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 20 weeks, at least about 25 weeks, at least about 30 weeks, at least about 35 weeks, at least about 40 weeks, at least about 45 weeks, at least about 50 weeks, at least about 52 weeks, at least about 13 months, at least about 14 months, at least about 15 months, or greater, including indefinitely.

### Glycemic Control and Hypomagnesemia

As described herein, certain subjects with PWS administered KAIP channel openers may demonstrate compromised glycemic control and of those, certain subjects were identified as having reduced serum magnesium levels. Glycemic control relates to the ability of a subject to manage an optimal blood glucose concentration. One way of measuring glycemic control is using a hemoglobin A1C (“HbA1c”) blood test, which measures the extent to which hemoglobin have sugar molecules attached to them as representative of average glucose levels of a subject over a period of time (up to 3 months). Subjects with PWS that demonstrate compromised glycemic control will likely present with increases in HbA1c levels with time. Poor or compromised glycemic control can present as prediabetes or diabetes mellitus and may result in worse treatment outcomes for subjects with PWS.

Magnesium (“Mg”) has a direct impact on glucose metabolism since it is a cofactor for numerous energy-metabolizing enzymes as well as a component of the Mg₂+-ATP complex. Magnesium is required for both aerobic and anaerobic energy synthesis via glycolysis and oxidative phosphorylation via the Mg₂+-ATP complex or directly as an enzyme opener. Magnesium is required for cellular glucose uptake and energy conversion, the autophosphorylation of the insulin receptor tyrosine kinase, and for the action of glucose transporter protein-4 (GLUT-4) which is critical for glucose uptake in muscle and fatty tissue. Hypomagnesemia has been associated with changes in cellular glucose transport, pancreatic insulin synthesis, post-receptor insulin signaling, and insulin receptor interaction. Normal function of all these processes is critical to maintenance of glycemic control.

As hypomagnesemia is characterized as a magnesium deficiency or lower-than-normal levels of serum magnesium, it can be appreciated that there are varying interpretations as to what qualifies as lower-than-normal levels of serum magnesium. While there is not consensus in the field regarding at what serum concentration of magnesium a subject can be considered to have hypomagnesia, as used herein, hypomagnesia refers to concentrations of serum magnesium less than 2.0 mg/dL. Other sources characterize hypomagnesia as less than 2.07 mg/dL (European Journal of Nutrition 2022; 61:3697-3706), 1.46 mg/dL (Gragossian A, Bashir K, Bhutta B S, et al. Hypomagnesemia. [Updated 2022 Nov. 4]. In: StatPearls), or 1.8 mg/dL (Lewis III, J. L., (2021). However, in this disclosure, hypomagnesemia may refer to when a subject's serum concentration of magnesium at the time of testing is less than 2.0 mg/dL. A subject with PWS may be considered to have a normal serum magnesium concentration when the subject with PWS has a serum magnesium concentration greater than 2.2 mg/dL. In some embodiments, a subject with PWS may have a serum magnesium concentration that is slightly below normal, wherein the subject with PWS may have a serum magnesium concentration greater than 2.0 mg/dL and below 2.2 mg/dL.

In some embodiments, the time of testing may refer to any point in time in which a subject's serum magnesium is measured. For example, in some embodiments, the time of testing may refer to the point in time before a magnesium supplement or a K_{ATP} channel opener is administered to a subject. The time of testing may also refer to any point in time after a magnesium supplement or a K_{ATP} channel opener is administered to a subject, as will be described in more detail herein.

### Magnesium Supplementation and Exemplary Magnesium Supplements

Since certain subjects with PWS administered a K_{ATP} channel opener may demonstrate hypomagnesemia which can lead to compromised glycemic control, these subjects with PWS may benefit from magnesium supplementation (e.g., the administration of a magnesium supplement) to boost serum magnesium levels, potentially improving the regulation of glycemic control (e.g., reducing hyperglycemia, which can be identified by elevated blood glucose concentrations).

In some embodiments, magnesium supplementation may include administering to a subject with PWS, a magnesium supplement. In some embodiments, a magnesium supplement may include a magnesium formulation or a formulation of a pharmaceutically acceptable salt of magnesium that may be administered to a subject with PWS in need thereof. In some embodiments, the magnesium supplement may include a magnesium formulation or a formulation of a pharmaceutically acceptable salt of magnesium in an amount useful for increasing the serum magnesium concentration in vivo. In some embodiments, the magnesium is in the form of magnesium oxide. It can be appreciated that one skilled in the art would be able to readily determine an amount of a formulation of magnesium or a corresponding amount of a pharmaceutically acceptable salt of magnesium for increasing the serum concentration in vivo. For example, useful amounts of magnesium, or a corresponding amount of a pharmaceutically acceptable salt of magnesium, can include, for example, about 100 mg to about 600 mg, including about 300 mg to about 500 mg of magnesium, or a corresponding amount of a pharmaceutically acceptable salt of magnesium. As used herein, recitation of magnesium doses (in milligrams) refers to the milligram amount of elemental magnesium in the magnesium supplement, unless clearly referencing a specific corresponding amount of a pharmaceutically acceptable salt of magnesium.

### Formulations of Magnesium and Pharmaceutically Acceptable Salts of Magnesium

In some embodiments, formulations of magnesium or pharmaceutically acceptable salts of magnesium can include for example, magnesium stearate, magnesium oxide, magnesium citrate, magnesium chloride, magnesium lactate, magnesium malate, magnesium taurate, magnesium glycinate, magnesium orotate, or mixtures of one or more of the foregoing.

A corresponding amount of a pharmaceutically acceptable salt of magnesium will be greater than the recited dose of magnesium. For example, a magnesium formulation containing 400 mg dose of magnesium oxide is a 241.3 mg dose of elemental magnesium. Similarly, a 400 mg dose of magnesium would require administration of approximately 663 mg of magnesium oxide. Calculation of the amount of a particular magnesium salt need to achieve a magnesium dose of a specified amount is readily determined by those of routine skill in the art.

### Frequency, Mechanism of Delivery, and Dosage Form of Exemplary Magnesium Supplements

In some embodiments, a magnesium supplement may be administered to the subject at least once per about 24 hours (e.g., once per 23-26 hours), for example one time per about 24 hours or two or more times per about 24 hours. For example, in some embodiments, the magnesium supplement may be administered to the subject once in the morning, once in the evening, or once in the morning and once in the evening. In some embodiments, the magnesium supplement may be administered to the subject three times per about 24 hours, four times per about 24 hours, five times per about 24 hours, 6 times per about 24 hours, 7 times per about 24 hours, or 8 times per about 24 hours. In some embodiments, if the magnesium supplement is administered to the subject two or more times per about 24 hours, the magnesium supplement may be administered to the subject a second time after a first period of time. In some embodiments, the first period of time may include about 1 hour, about 2 hours, about 3 hours, about 4 hours, about 5 hours, about 6 hours, about 7 hours, about 8 hours, about 9 hours, about 10 hours, about 11 hours, or about 12 hours. For example, the magnesium supplement may be first administered to the subject, and then after about a 6-hour period of time, the magnesium supplement may be administered to the subject for a second time.

The magnesium supplement may be administered to the subject for any length of time greater than or equal to about 1 week. For example, the magnesium supplement may be administered to the subject for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 20 weeks, at least about 25 weeks, at least about 30 weeks, at least about 35 weeks, at least about 40 weeks, at least about 45 weeks, at least about 50 weeks, at least about 52 weeks, at least about 13 months, at least about 14 months, at least about 15 months, or greater, including indefinitely.

In general, the length of time for administration of the magnesium supplement will continue as long as the K_{ATP} channel opener is administered to the subject with PWS. In some embodiments, the administration of the magnesium supplement will begin at the same time as when the K_{ATP} channel opener is first administered. In some embodiments, after the K_{ATP} channel opener is first administered, the administration of the magnesium supplement will begin about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 20 weeks, about 25 weeks, about 30 weeks, about 35 weeks, about 40 weeks, about 45 weeks, about 50 weeks, or about 52 weeks after the K_{ATP} channel opener is first administered. In some embodiments, the administration of the magnesium supplement begins within about 0 to about 3, about 0 to about 4, about 0 to about 6, about 0 to about 12, or about 0 to about 24 months after the first administration of the K_{ATP} channel opener. In some embodiments, the administration of the magnesium supplement begins within about 1 to about 3, about 1 to about 4, about 1 to about 6, about 1 to about 12, about 1 to about 24, about 2 to about 3, about 3 to about 4, about 3 to about 6, about 3 to about 12, about 3 to about 24, about 2 to about 3, about 2 to about 4, about 3 to about 6, about 4 to about 12, or about 4 to about 24 months after the first administration of the K_{ATP} channel opener.

In some embodiments, the magnesium supplement may be administered to the subject using various mechanisms of delivery including, but not limited to, oral delivery, sublingual delivery, intravenous delivery, or the like. In some embodiments, the magnesium supplement may be administered to the subject using the same mechanism of delivery for each administration or different mechanisms of delivery for each administration. In certain embodiments, the magnesium supplement is administered orally, for example, in a solid oral dosage form such as one or more tablets or capsules.

In some embodiments, the magnesium supplement may be administered to the subject as one or more dosage forms. The one or more dosage forms may include liquid (e.g., such as a suspension), solid, or semi-solid dosage forms. For example, in some embodiments, the one or more dosage forms may include a tablet, a capsule, a powder, a solution, a suspension, a paste, or the like. In some embodiments, the magnesium formulation or the pharmaceutically acceptable salt of magnesium may be administered to the subject with the same dosage form for every administration. In other embodiments, the magnesium supplement may be administered to the subject with different dosage forms for each administration. For example, in some embodiments, the subject may be administered a tablet containing the magnesium formulation for each administration. In other embodiments, the subject may be administered first, a tablet containing the magnesium formulation and second, a solution containing the magnesium formulation.

In some embodiments, the magnesium supplement may include one or more excipients. The one or more excipients may include diluents, binders, compression aids, granulating agents, disintegrants, glidants, lubricants, tablet coatings and films, coloring agents, or the like.

### Daily Dose of Magnesium Supplement

In some embodiments, the subject may be administered a desired total daily dose of the magnesium supplement. In some embodiments, the desired total daily dose may vary based on a subject's serum magnesium concentration at the time of testing. In some embodiments, the desired total daily dose may not vary and may be consistent across all subjects regardless of the subject's serum magnesium concentration at the time of testing. For example, in subjects demonstrating hypomagnesemia, the desired total daily dose may be greater than a subject demonstrating a slightly lower-than-normal serum magnesium concentration. However, in some embodiments, all subjects regardless of serum magnesium concentration may derive benefit from a desired total daily dose of magnesium supplement greater than about 300 mg. In some embodiments, hypomagnesemia may occur when a subject with PWS has a serum magnesium concentration of less than 2.0 mg/dL. In some embodiments, a subject with PWS may have a normal serum magnesium concentration that is normal when the subject with PWS has a serum magnesium concentration greater than 2.2 mg/dL. In some embodiments, a subject with PWS may have a serum magnesium concentration that is slightly below normal, wherein the subject with PWS may have a serum magnesium concentration greater than 2.0 mg/dL and below 2.2 mg/dL.

In some embodiments, the subject may be administered the magnesium supplement until the subject is administered a desired total daily dose. For example, for a subject to obtain a desired daily dose of 500 mg of magnesium, a single dosage form (e.g., tablet, capsule, etc.) containing 500 mg of a magnesium formulation or two or more dosage forms (e.g., a 200 mg tablet and a solution having 300 mg of magnesium therein) comprising a total of 500 mg of the magnesium formulation can be administered to the subject. As would be appreciated by the skilled artisan, additional combinations of dosage forms with varying amounts of magnesium, or a pharmaceutically acceptable salt of magnesium, may be administered one or more times per 24 hours to achieve the desired magnesium supplement daily dose.

Administration of Magnesium with KAIP Channel Opener

In some embodiments, the magnesium supplement may be administered to the subject with PWS at substantially the same time (e.g., co-administered) as the K_{ATP} channel opener. In some embodiments, co-administration may include the administration of two doses of two separate medications in a close proximity of time (e.g., within 5-10 minutes of each other) via separate dosage forms or via a single combined dosage form. For example, the magnesium supplement in a capsule form may be orally administered to the subject with PWS right before or after the K_{ATP} channel opener (e.g., extended-release diazoxide choline in tablet form) is administered to the subject with PWS. In some embodiments, the magnesium supplement may be combined with the K_{ATP} channel opener into a single dosage form.

In some embodiments, the K_{ATP} channel opener may be administered to the subject with PWS for the same duration of time as the administration of the magnesium supplement or an extended duration of time before or after the administration of the magnesium supplement. For example, the K_{ATP} channel opener may be administered to the subject with PWS about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 20 weeks, about 25 weeks, about 30 weeks, about 35 weeks, about 40 weeks, about 45 weeks, about 50 weeks, or about 52 weeks or more before the administration of the magnesium supplement. In some embodiments, the administration of the K_{ATP} channel opener begins within about 0 to about 3, about 0 to about 4, about 0 to about 6, about 0 to about 12, or about 0 to about 24 months before the first administration of the magnesium supplement. In some embodiments, the administration of the K_{ATP} channel opener begins within about 1 month to about 3 months, about 1 month to about 4 months, about 1 month to about 6 months, about 1 month to about 12 months, about 1 month to about 24 months, about 2 months to about 3 months, about 3 months to about 4 months, about 3 months to about 6 months, about 3 months to about 12 months, about 3 months to about 24 months, about 2 months to about 3 months, about 2 months to about 4 months, about 3 months to about 6 months, about 4 months to about 12 months, or about 4 months to about 24 months.

Subjects with PWS in Need Thereof

It can be appreciated that only certain subjects with PWS may be in need of the administration of a magnesium supplement to help manage glycemic control. In some embodiments, subjects with PWS that may be in need of administration of a magnesium supplement include subjects with PWS demonstrating, or at risk for, hypomagnesemia. In some embodiments, due to one or more risk factors, some subjects with PWS may be at greater risk for hypomagnesemia compared to other subjects with PWS lacking the one or more risk factors. In some embodiments, the one or more risk factors may include, but are not limited to, age, obesity status, having diabetes mellitus or presenting as pre-diabetic, PWS genetic subtype(s), or a serum concentration of magnesium less than or equal to 2.2 mg/dL or the like, as will be described in more detail herein.

In some embodiments, one risk factor for hypomagnesemia in a subject with PWS is age. Subjects with PWS older than about 17 years of age may be at increased risk for hypomagnesemia compared to subjects with PWS less than 17 years of age. In some embodiments, another risk factor for hypomagnesemia in a subject with PWS is obesity status. Subjects with PWS that are obese may be at an increased risk for hypomagnesemia compared to subjects with PWS that are not obese.

Some subjects with PWS may have a Type 1 deletion in the 15q11.2-q13 chromosomal region, encompassing breakpoints BP1 to BP3 in Chromosome 15. Type 1 deletions can lead to loss of some copies of magnesium transport genes. Therefore, subjects with PWS that have Type 1 deletions may be at an increased risk for hypomagnesemia compared to subjects with PWS not having Type 1 deletions (i.e., with other genetic subtypes).

In some embodiments, increased risk of diabetes mellitus and/or presenting as pre-diabetic may increase the risk for subjects with PWS to have hypomagnesemia. Additionally, in subjects with PWS, the risk of being diagnosed as pre-diabetic or having diabetes may increase with age from adolescence to adulthood, further increasing the risk of hypomagnesemia.

Additionally, subjects with PWS having a serum concentration of magnesium less than or equal to 2.2 mg/dL at the time of testing may be at an increased risk for hypomagnesemia compared to PWS subjects having a serum concentration of magnesium greater than 2.2 mg/dL at the time of testing.

Methods of Managing Glycemic Control in a Subject with PWS in Need Thereof

Also disclosed herein are methods of managing glycemic control in a subject with PWS in need thereof. In some embodiments, the method comprises co-administering daily to the subject with PWS in need thereof, (i) a daily dose of a K_{ATP} channel opener, or a pharmaceutically acceptable salt thereof, and (ii) a daily dose of a magnesium supplement for at least 1 week. In some embodiments, as described above, the daily dose of the K_{ATP} channel opener may include a dose of about 15 mg to about 400 mg, or about 25 mg to about 580 mg. In some embodiments, as described above, the daily dose of the magnesium supplement may include a daily dose of about 100 mg to about 600 mg.

As described above, the daily co-administration to the subject with PWS in need thereof of a daily dose of a K_{ATP} channel opener or a pharmaceutically acceptable salt thereof and a daily dose of a magnesium supplement may occur for at least 1 week. In some embodiments, at least 1 week includes about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 20 weeks, about 25 weeks, about 30 weeks, about 35 weeks, about 40 weeks, about 45 weeks, about 50 weeks, about 52 weeks, or indefinitely.

In some embodiments, daily co-administration of a daily dose of a K_{ATP} channel opener and a daily dose of a magnesium supplement to a subject with PWS in need thereof may lead to measurable changes in the subject with PWS. For example, in some embodiments, the concentration of hemoglobin A1C (HbA1c) of the subject with PWS in need may decrease compared to the concentration of HbA1c of the subject with PWS as measured for the subject prior to commencing daily co-administration of a daily dose of a K_{ATP} channel opener and a daily dose of a magnesium supplement. In some embodiments, the concentration of HbA1c of the subject with PWS may decrease by at least about 0.5%, at least about 0.6%, at least about 0.7%, at least about 0.8%, at least about 0.9%, at least about 1.0%, or the like. For example, the concentration of HbA1c of the subject with PWS may decrease by between about 0.5% and 1.0%, including between about 0.6% and 0.9%, or between about 0.7% and 0.8% after daily co-administration of a daily dose of a K_{ATP} channel opener and a daily dose of a magnesium supplement to the subject with PWS in need thereof.

In some embodiments, daily co-administration of the daily dose of a K_{ATP} channel opener and the daily dose of the magnesium supplement to a subject with PWS in need thereof may lead to effective maintenance of glycemic control despite K_{ATP} channel opener induced reductions in insulin secretion as evidenced by very minor changes in HbA1c compared to before daily co-administration of the K_{ATP} channel opener and the daily dose of the magnesium supplement were commenced. As used herein, the term “minor changes” in HbA1c may include decreases in measured levels of HbA1c of about 0.01% to about 0.09% in a subject with PWS. This maintenance of glycemic control compared to before daily co-administration of the K_{ATP} channel opener and the daily dose of the magnesium supplement were commenced may be identified, in some embodiments, as a lessening of the increase of HbA1c week to week or month to month versus the rate of increase observed in the time periods before co-administration commences, particularly in at-risk subjects where co-administration of the magnesium supplement is at or proximate in time to the beginning of treatment with the K_{ATP} channel opener. In some embodiments, no change in HbA1c levels in a subject with PWS in need thereof may indicate the maintenance of glycemic control. In some embodiments, no change in HbA1c levels in a subject with PWS in need thereof may indicate that HbA1c is maintained within normal levels, despite administration with a K_{ATP} channel opener.

In some embodiments, daily co-administration of a daily dose of a K_{ATP} channel opener and a daily dose of a magnesium supplement to a subject with PWS in need thereof may lead to measurable changes in serum magnesium concentration in the subject with PWS. For example, in some embodiments, daily co-administration of a daily dose of a K_{ATP} channel opener and a magnesium supplement may lead to an increase in a PWS subject's serum magnesium concentration compared to the subject with PWS's serum magnesium concentration as measured for the subject prior to commencing co-administration of the daily dose of the K_{ATP} channel opener and a magnesium supplement. For example, co-administration of the daily dose of the K_{ATP} channel opener and a magnesium supplement for at least 1 week may lead to an increase in a subject with PWS's serum magnesium concentration greater than about at least 1.7 mg/dL, including greater than about at least 1.8 mg/dL, 1.9 mg/dL, 2.0 mg/dL, 2.07 mg/dL, 2.1 mg/dL, or 2.2 mg/dL. In some embodiments, the co-administration of the daily dose of the K_{ATP} channel opener and a magnesium supplement for at least 1 week may lead to the subject with PWS's serum magnesium concentration being greater than between about 1.7 mg/dL and 2.2 mg/dL including about 1.8 mg/dL and about 2.1 mg/dL, about 1.9 mg/dL and about 2.0 mg/dL. In some embodiments, the co-administration of the daily dose of the K_{ATP} channel opener and a magnesium supplement for at least 1 week may lead to the PWS subject's serum magnesium concentration being greater than 2.2 mg/dL including for example between about 2.2 mg/dL and about 2.6 mg/dL, about 2.3 mg/dL and about 2.5 mg/dL.

### Exemplary Embodiments

Some embodiments of this disclosure relate to Embodiment I, as follows:

Embodiment I-1 A method of managing glycemic control in a subject with Prader-Willi Syndrome in need thereof, comprising co-administering daily to the subject with Prader-Willi Syndrome in need thereof,
(i) a daily dose of a KAIP channel opener, or a pharmaceutically acceptable salt thereof, and
(ii) a daily dose of a magnesium supplement, for at least 1 week.
   Embodiment I-2 The method of Embodiment I-1, wherein the K_{ATP} channel opener, or pharmaceutically acceptable salt thereof, is diazoxide choline.
   Embodiment I-3 The method of Embodiment I-1 or Embodiment I-2, wherein administration is daily for at least 4 weeks.
   Embodiment I-4 The method of Embodiment I-3, wherein the administration is daily for at least 8 weeks.
   Embodiment I-5 The method of Embodiment I-4, wherein the administration is daily for at least 13 weeks.
   Embodiment I-6 The method of Embodiment I-5, wherein the duration of administration is daily for at least 1 year.
   Embodiment I-7 The method of any of Embodiment I-1 to Embodiment I-6, wherein a total daily dose of the K_{ATP} channel opener is about 25 mg to about 585 mg, or a corresponding amount of a pharmaceutically acceptable salt thereof.
   Embodiment I-8 The method of any of Embodiment I-1 to Embodiment I-7, wherein the total daily dose of the magnesium supplement comprises a total daily dose of magnesium of about 100 mg to about 600 mg, or a corresponding amount of a pharmaceutically acceptable salt or mixture of magnesium.
   Embodiment I-9 The method of Embodiment I-8, wherein the total daily dose of magnesium is about 250 mg to about 500 mg, or a corresponding amount of a pharmaceutically acceptable salt or mixture of magnesium.
   Embodiment I-10 The method of any one of Embodiment I-1 to Embodiment I-9, wherein the total daily dose of the K_{ATP} channel opener, or pharmaceutically acceptable salt thereof, is administered once daily.
   Embodiment I-11 The method of any one of Embodiment I-1 to Embodiment I-10, wherein the total daily dose of the K_{ATP} channel opener, or pharmaceutically acceptable salt thereof, is formulated as an extended-release formulation.
   Embodiment I-12 The method of any one of Embodiment I-1 to Embodiment I-11, wherein the total daily dose of the magnesium supplement is administered once daily.
   Embodiment I-13 The method of any one of Embodiment I-1 to Embodiment I-12, wherein the total daily dose of magnesium supplement and the daily dose of K_{ATP} channel opener, or pharmaceutically acceptable salt thereof, are each administered once daily.
   Embodiment I-14 The method of any one of Embodiment I-1 to Embodiment I-12, wherein the total daily dose of K_{ATP} channel opener, or pharmaceutically acceptable salt thereof, is administered once daily and the magnesium supplement is administered two or more times daily.
   Embodiment I-15 The method of any of Embodiment I-1 to Embodiment I-14, wherein the total daily dose of each of the K_{ATP} channel opener, or pharmaceutically acceptable salt thereof, and/or the magnesium supplement, is administered as two or more dosage forms.
   Embodiment I-16 The method of any one of Embodiment I-1 to Embodiment I-15, wherein the magnesium supplement is a solid dosage form.
   Embodiment I-17 The method of any one of Embodiment I-1 to Embodiment I-16, wherein the subject with Prader-Willi Syndrome in need thereof is at risk for hypomagnesemia.
   Embodiment I-18 The method of Embodiment I-17, wherein the subject with Prader-Willi Syndrome at risk for hypomagnesemia is about 17 years of age or older.
   Embodiment I-19 The method of Embodiment I-17 or Embodiment I-18, wherein the subject with Prader-Willi Syndrome at risk for hypomagnesemia is obese.
   Embodiment I-20 The method of any one of Embodiment I-1 to Embodiment I-19, wherein the subject with Prader-Willi Syndrome in need thereof has a Type 1 deletion within 15q11.2-q13 encompassing breakpoints BP1 to BP3 in Chromosome 15.
   Embodiment I-21 The method of any claims of Embodiment I-1 to Embodiment I-20, wherein the subject with Prader-Willi Syndrome in need thereof has a serum magnesium concentration threshold of less than about 2.2 mg/dL prior to treatment.
   Embodiment I-22 The method of Embodiment I-21, wherein the subject with Prader-Willi Syndrome in need thereof has a serum magnesium concentration of less than about 2.0 mg/dL prior to treatment.
   Embodiment I-23 The method of any one of Embodiment I-1 to Embodiment I-22, wherein the subject with Prader-Willi Syndrome's hemoglobin A1C (HbA1c), decreases by at least 0.5%, compared to prior to treatment.
   Embodiment I-24 The method of Embodiment I-23, wherein the subject with Prader-Willi Syndrome's HbA1c decreases by at least 0.7%, compared to prior to treatment.
   Embodiment I-25 The method of Embodiment I-24, wherein the subject with Prader-Willi Syndrome's HbA1c decreases by at least 0.8%, compared to prior to treatment.
   Embodiment I-26 The method of any one of Embodiment I-1 to Embodiment I-25, wherein the subject with Prader-Willi Syndrome's serum magnesium concentration after at least 1 week of treatment is greater than about at least 1.7 mg/dL.
   Embodiment I-27 The method of any one Embodiment I-1 to Embodiment I-25, wherein subject with Prader-Willi Syndrome's serum magnesium concentration after at least 1 week of treatment is greater than about at least 2.00 mg/dL.
   Embodiment I-28 Use of the magnesium supplement of Embodiment I-I in the manufacture of a medicament for managing glycemic control in a subject with Prader-Willi Syndrome in need thereof.
   Embodiment I-29 The method of Embodiment I-I, wherein daily co-administration of the daily dose the K_{ATP} channel opener, or a pharmaceutically acceptable salt thereof, and a daily dose of the magnesium supplement to the subject with Prader-Willi Syndrome in need thereof, includes effective maintenance of glycemic control in the subject with Prader-Willi Syndrome.
   Embodiment I-30 The method of Embodiment I-29, wherein effective maintenance of glycemic control in the subject with Prader-Willi Syndrome includes decrease of about 0.01% to about 0.09% in measured levels of hemoglobin A1C (“HbA1c”) in the subject with Prader-Willi Syndrome compared to prior to treatment.

### EXAMPLES

The following specific examples are to be construed as merely illustrative, and not limitative of the remainder of the disclosure in any way whatsoever.

### Example 1: Exemplary Formulations of an Extended-Release K_{ATP} Channel Opener

This example describes exemplary extended-release formulations of diazoxide choline. In some embodiments, exemplary extended-release formulations of diazoxide choline may be as described in Tables 1.0 and 1.1. Additional exemplary formulations of diazoxide may be described in U.S. Pat. Nos. 7,799,777, 7,572,789, 9,381,202, 10,085,998, 11,045,478, 9,757,384, 10,058,557, and 10,456,408, each of which is incorporated by reference in its entirety herein. In some embodiments, the formulations may include one or more of: a hydroxypropyl methylcellulose e.g., “Methocel® K100M” as supplied by DuPont, a colloidal silicon dioxide e.g., “Cab-o-sil” as supplied by Cabot Corporation, a mixture of polyvinyl acetate and povidone e.g., “Kollidon® SR” as supplied by BASF, a sodium stearyl fumarate e.g., “Pruv®” as supplied by JRS Pharma, a poly(ethylene oxide) “PEO” polymer e.g., POLYOX™ WSR Coagulant N750 NF or N303 NF as supplied by DuPont, calcium phosphates e.g., “EMCOMPRESS®” as supplied by JRS Pharma or talcum (“talc”).

TABLE 1.0
Exemplary Formulations for Diazoxide Choline

| | | Weight % of total |
|---|---|---|
| Formulation | INGREDIENT | composition weight |
| A. | Diazoxide Choline | 29.0 |
| | KOLLIDON® SR | 40.0 |
| | PEO N303 | 10 |
| | EMCOMPRESS® | 20.2 |
| | PRUV® | 0.8 |
| | % TOTAL WEIGHT | 100 |
| B. | Diazoxide Choline | 20.7 |
| | METHOCEL® K100M | 67.8 |
| | EMCOMPRESS® | 10.0 |
| | Magnesium Stearate | 1 |
| | Cab-o-sil | 0.5 |
| | TOTAL WEIGHT | 100 |
| C. | Diazoxide Choline | 20.7 |
| | KOLLIDON® SR | 30.0 |
| | PEO N303 | 10.0 |
| | EMCOMPRESS® | 38.3 |
| | PRUV® | 1 |
| | TOTAL WEIGHT | 100 |
| D. | Diazoxide Choline | 29.0 |
| | METHOCEL® K100M | 40.0 |
| | EMCOMPRESS® | 10.0 |
| | Magnesium Stearate | 20.2 |
| | Cab-o-sil | 0.8 |
| | TOTAL WEIGHT | 100.0 |
| E. | Diazoxide Choline | 20.7 |
| | PEO N750 NF | 67.8 |
| | PEO N303 NF | 10.0 |
| | EMCOMPRESS® | 1.0 |
| | PRUV® | 0.5 |
| | TOTAL WEIGHT | 100.0 |
| F. | Diazoxide Choline | 20.7 |
| | KOLLIDON® SR | 30.0 |
| | PEO N303 | 10.0 |
| | EMCOMPRESS® | 38.3 |
| | PRUV® | 1.0 |
| | TOTAL WEIGHT | 100.0 |
| G. | Diazoxide Choline | 20.7 |
| | METHOCEL® K100M | 50.0 |
| | EMCOMPRESS® | 27.8 |
| | Magnesium Stearate | 1.0 |
| | Cab-o-sil | 0.5 |
| | TOTAL WEIGHT | 100.0 |
| H. | Diazoxide Choline | 20.7 |
| | PEO N750 NF | 30.0 |
| | PEO N303 NF | 15.0 |
| | EMCOMPRESS® | 33.3 |
| | PRUV® | 1.0 |
| | TOTAL WEIGHT | 100.0 |
| I. | Diazoxide Choline | 20.7 |
| | METHOCEL® K100M | 30.0 |
| | EMCOMPRESS® | 2.0 |
| | Magnesium Stearate | 46.3 |
| | Cab-o-sil | 1.0 |
| | TOTAL WEIGHT | 100.0 |
| J. | Diazoxide Choline | 20.4 |
| | PEO N750 NF | 29.5 |
| | PEO N303 NF | 14.8 |
| | EMCOMPRESS® | 32.8 |
| | PRUV® | 1.0 |
| | Talc | 1.0 |
| | Cab-o-sil | 0.5 |
| | TOTAL WEIGHT | 100.0 |
| K. | Diazoxide Choline | 35.0 |
| | PEO N750 NF | 30.0 |
| | PEO N303 NF | 15.0 |
| | EMCOMPRESS® | 17.5 |
| | PRUV® | 1.0 |
| | Talc | 1.0 |
| | Cab-o-sil | 0.5 |
| | TOTAL WEIGHT | 100.0 |
| L. | Diazoxide Choline | 20.7 |
| | PEO N750 NF | 30.0 |
| | PEO N303 NF | 15.0 |
| | EMCOMPRESS® | 31.8 |
| | PRUV® | 0.1 |
| | Talc | 1.0 |
| | Cab-o-sil | 0.5 |
| | TOTAL WEIGHT | 100.0 |

TABLE 1.1
An Exemplary Formulation for Diazoxide Choline

| | Weight % of total |
|---|---|
| INGREDIENT | composition weight |
| Formulation M. | |
| Diazoxide Choline | 8.80-26.40 |
| Polyethylene Oxide (WSR N750) | 30.00 |
| Polyethylene Oxide (WSR 303) | 15.00 |
| Silicified Microcrystalline Cellulose | 19.10 |
| Dibasic Calcium Phosphate, Dihydrate, | 5.0-23.60 |
| Talc, USP/EP | 1.33-2.00 |
| Colloidal Silicon Dioxide | 0.667-1.00 |
| Magnesium Stearate, NF | 1.5 |

### Example 2: Reductions in Serum Magnesium Account for Much of the Worsened Glycemic Control in PWS Subjects Administered Extended-Release Diazoxide Choline

A multi-center international, randomized, double-blind, placebo-controlled, parallel-group, phase 3 study has been carried out to compare administration of extended-release diazoxide choline to placebo in individuals with PWS. The study enrolled males and females with genetically confirmed PWS, aged 4 years and older with moderate to severe hyperphagia (Hyperphagia Questionnaire for Clinical Trials [HQ-CT] score ≥13), weighing between 20 and 135 kg, in a stable care setting (no change in care setting for 6 months prior to enrollment and no planned change during the study). Growth hormone treatment of children and adults was permitted. The enrolled subjects were subjected to measurement of key endpoints to determine baseline measurements and were randomly assigned 2:1 to treatment with extended-release diazoxide choline or matching placebo stratified by growth hormone treatment (currently treated, not currently treated) and baseline HQ-CT score (scores from 13-19 and from 20-36). Participants were included in 1 of 5 weight bands (20 to <30 kg; ≥30 to <40 kg; ≥40 to <65 kg; ≥65 to <100 kg; and ≥100 to <135 kg) and titrated to target dose of 3.3 to 5.8 mg/kg within 6 weeks. Daily dose of extended-release diazoxide choline ranged from 100 to 450 mg. Multiple endpoints were assessed including hyperphagia change from baseline through week 13 measured using the HQ-CT (a 9-item, validated questionnaire that assessed food-related behaviors associated with hyperphagia in people with PWS with a range of 0-36), Clinical Global Impression of Improvement (CGI-I), Caregiver Global Impression of Change (GI-C), body fat measured using dual-energy x-ray absorptiometry, other body composition parameters, behavioral complications of PWS assessed using the Prader-Willi Syndrome Profile (PWSP) Questionnaire and the Developmental Behavior Checklist version 2 parent edition (DBC2-P), and hormones involved in appetite, satiety, and fat metabolism (e.g., leptin, adiponectin, insulin, and acylated ghrelin), were all measured at a central laboratory. The Homeostatic Model Assessment of Insulin Resistance (HOMA-IR) was calculated using fasting insulin and glucose measurements from the central laboratory as [fasting insulin (μIU/mL)×fasting glucose (mmol/L)]/22.5. HbA1c was also measured. The primary efficacy endpoint was analyzed using a linear mixed model for repeated measurements including fixed effects for time, using baseline HQ-CT score as a covariate and an unstructured covariance model. The analysis included all available data from all participants. Hormonal, endocrine, body composition, and behavioral parameters were analyzed using the same approach as the primary endpoint, with the baseline value for the parameter as a covariate. Caregiver Global Impression of Severity and Clinical Global Impression of Severity were analyzed using the Wilcoxon signed rank test.

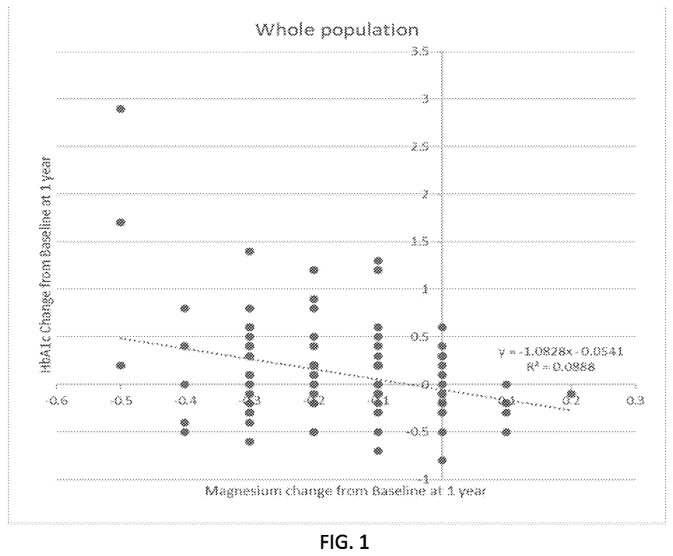

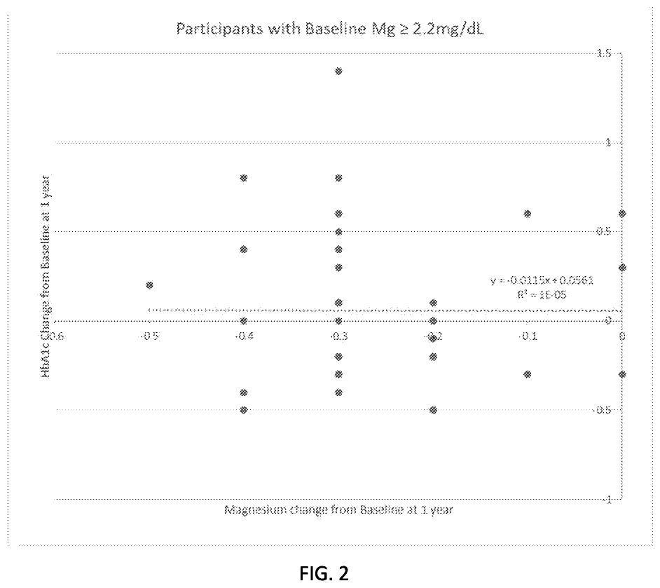

From this data set acquired from the multi-center international, randomized, double-blind, placebo-controlled, parallel-group, phase 3 study described above, a data set corresponding to a time period of 1 year of KAIP channel opener administration was assembled using the last pre-extended-release diazoxide choline measurement as a baseline value and the data closest to 1 year of administration date as a response parameter. In some embodiments, the data closest to 1 year of administration date may have been collected at a study visit or from an unscheduled measurement. The 1-year data set was analyzed, comparing the changes in HbA1c from baseline and 1 year of administration of extended-released diazoxide choline with changes in magnesium measurements from baseline to 1 year of administration of extended-released diazoxide choline. FIG. 1 depicts a graph of the analyzed data of the entire cohort of PWS subjects comparing the changes in HbA1c from baseline and 1 year of administration of extended-released diazoxide choline with changes in magnesium measurements from baseline to 1 year of administration of extended-released diazoxide choline. FIGS. 2-7 depict graphs of the analyzed data of sub-populations of subjects with PWS, comparing the changes in HbA1c from baseline and 1 year of administration of extended-released diazoxide choline with changes in magnesium measurements from baseline to 1 year of administration of extended-released diazoxide choline, as will be described in more detail herein.

Graphs from FIGS. 2-7 depict Pearson correlation coefficients calculated using all of the available measurements of HbA1c and magnesium levels, including at each of baseline (124 pairs of values; non-significant) and at 52 weeks (105 pairs of values; p<0.001). The Pearson correlation coefficient between change in HbA1c and change in magnesium levels was also significant (p<0.01).

At baseline, wherein baseline refers to any value measured before the first administration of the K_{ATP} channel opener, there was no correlation between HbA1c and magnesium (r=−0.0367), data not shown. After one year of administration of K_{ATP} channel opener, there was a significant negative correlation between HbA1c and magnesium (r=−0.507). This may have resulted from a limited number of individuals with large reductions in magnesium and corresponding large increases in HbA1c. The correlation between HbA1c change from baseline and magnesium change from baseline was −0.299, indicating a slight negative relationship.

Changes in Concentration of Magnesium and Changes in Concentration of HbA1c in the Population of Subjects with PWS

There appeared to be a small reduction in magnesium through 15 months of administration of the K_{ATP} channel opener. Similarly, HbA1c values across the population of subjects with PWS rose from about 26 to about 39 weeks, and HbA1c values began to drop from about 39 weeks through about 65 weeks. At baseline, most subjects with PWS had magnesium levels greater than about 2.0 mg/dL. However, after 1 year of K_{ATP} channel opener administration, approximately 6 of 101 subjects showed an increase in magnesium while another 11 subjects with PWS showed no change in magnesium. Strikingly, eighty-four percent of the population of subjects with PWS showed a reduction in serum magnesium, with a few subjects with PWS demonstrating drastic reductions in serum magnesium that put them in a state of hypomagnesemia. This indicates that long term administration of a K_{ATP} channel opener (e.g., extended-released diazoxide choline) may lead do decreases in magnesium in subjects with PWS, potentially leading to a decrease in glycemic control.

### Example 2.1 Subjects with PWS Demonstrate a Negative Relationship Between Changes in Concentration of Magnesium and Changes in Concentration of HbA1c

There was a negative relationship between changes in measured serum magnesium levels and changes in measured HbA1c for subjects with PWS administered extended-release diazoxide choline for 1 year as seen in FIG. 1. Subjects with PWS with higher HbA1c changes from baseline to the 1-year mark demonstrate low magnesium serum changes from baseline to one year. However, the population of subjects with PWS can be broken down into sub-populations. For example, the population of subjects with PWS can be broken down into subjects with PWS demonstrating serum magnesium levels above a measured threshold, subjects with PWS demonstrating serum magnesium levels below the measured threshold, subjects with PWS presenting with a Type I deletion subtype, subjects with PWS not presenting with a Type I deletion subtype, and subjects with PWS presenting as obese. Each of these sub-populations can be analyzed to determine if a relationship exists between changes in measured serum magnesium levels and changes in measured HbA1c for subjects with PWS administered extended-release diazoxide choline for 1 year.

### Example 2.2 Subjects with PWS with Baseline Measured Serum Magnesium Concentrations Greater than 2.2 mg/dL Demonstrate No Relationship Between Changes in Concentration of HbA1c and Changes in Serum Magnesium Concentration after Administration of K_{ATP} Channel Opener

The population of subjects with PWS was narrowed from the whole population to interrogate subjects with PWS administered extended-release diazoxide choline for 1 year and having a baseline measured serum magnesium level greater than 2.2 mg/dL. FIG. 2 depicts a graph of changes in measured serum magnesium levels and changes in measured HbA1c for subjects with PWS administered extended-release diazoxide choline for 1 year. No relationship was established between changes in magnesium and changes in HbA1c. For example, as seen in FIG. 2, change in HbA1c and change in serum magnesium was essentially 0 and R² for the regression was 1.5e-05, also essentially 0 as indicated by the dotted line. In this subgroup, the average change in HbA1c was 0.07% and the largest change in HbA1c was 0.8 in a participant that was diabetic at baseline.

### Example 2.3 Subjects with PWS with Baseline Measured Serum Magnesium Concentrations Between 2.0 and 2.2 mg/dL Demonstrate a Trend of a Negative Relationship Between Changes in Concentrations of Serum Magnesium and Changes in HbA1c after Administration of K_{ATP} Channel Opener

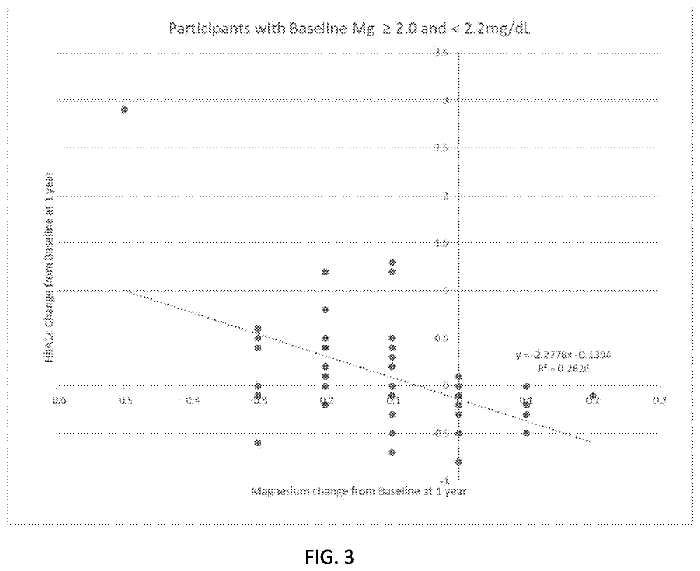

The population of subjects with PWS was narrowed from the whole population to interrogate subjects with PWS administered extended-release diazoxide choline for 1 year and having a baseline measured serum magnesium level between 2.0 and 2.2 mg/dL. Subjects with PWS having a baseline measured serum magnesium level closer to 2.0 are considered to be trending towards hypomagnesemia. FIG. 3 depicts a graph of changes in measured serum magnesium levels and changes in measured HbA1c for PWS subjects administered extended-release diazoxide choline for 1 year. In participants with magnesium levels 2 mg/dL to ≤2.2 mg/dL (the middle of the normal range), there was a trend towards increases in HbA1c associated with reductions in magnesium and the participant with the greatest change in magnesium also showed the greatest increase in HbA1c (FIG. 3).

### Example 2.4 Subjects with PWS with Hypomagnesemia Demonstrate a Strong Negative Relationship Between Changes in Concentrations of Serum Magnesium and HbA1c after Administration of K_{ATP} Channel Opener

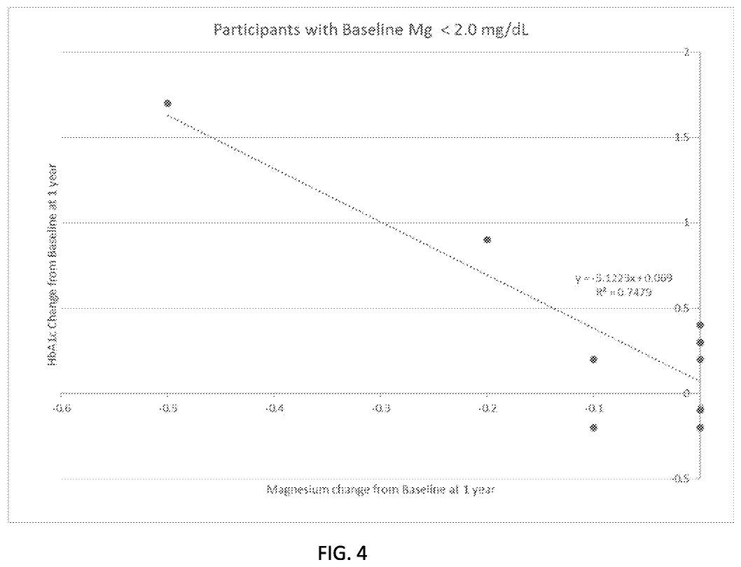

The population of subjects with PWS was narrowed from the whole population to interrogate subjects with PWS administered extended-release diazoxide choline for 1 year and having a baseline measured serum magnesium level less than 2.0 mg/dL. Subjects with PWS having a baseline measured serum magnesium level less than 2.0 mg/dL are considered to have hypomagnesemia. FIG. 4 depicts a graph of changes in measured serum magnesium levels and changes in measured HbA1c for subjects with PWS administered extended-release diazoxide choline for 1 year. For subjects with PWS having a baseline measured serum magnesium level less than 2.0 mg/dL, there was a strong negative relationship between changes in serum magnesium and changes in HbA1c after 1 year of administration of extended-release diazoxide choline. For subjects with PWS demonstrating hypomagnesemia, there was a much stronger negative relationship between serum magnesium changes and changes in HbA1c than for subjects with PWS demonstrating baseline serum magnesium levels between 2.0 and 2.2 mg/dL. This suggests that the effect of changes in serum magnesium on HbA1c depends on the baseline magnesium level. Put simply, subjects with PWS having measured baseline serum magnesium concentrations greater than 2.2 mg/dL, will likely not demonstrate serum magnesium levels less than 2.2 mg/dL after 1 year of extended-release diazoxide choline administration. Subjects with PWS having measured baseline serum magnesium levels between 2.0 and 2.2 mg/dL, may demonstrate a decrease in serum magnesium levels after 1 year of extended-release diazoxide choline administration. Subjects with PWS having hypomagnesemia (e.g., baseline serum magnesium levels less than 2.0 mg/dL) will likely demonstrate hypomagnesemia after 1 year of extended-release diazoxide choline administration.

### Example 2.5 Subjects with PWS Having a Type 1 Deletion Demonstrate a Strong Negative Relationship Between Changes in Concentration of Serum Magnesium and HbA1c Compared to Subjects with PWS Having Non-Type 1 Deletion after Administration of K_{ATP} Channel Opener

There are two types of classes of typical deletions seen in subjects with PWS, Type 1 and Type 2. Type 1 deletions are larger and involve a proximal breakpoint BP1, nearer to the centromere than type 2 deletions seen at a proximal breakpoint BP2. The Type 1 deletions lead to loss of copies of paternal genes including some magnesium transport genes, thus potentially giving subjects with PWS having a Type 1 deletion a pre-disposition to lower serum magnesium levels than subjects with PWS without the Type 1 deletion subtype. Additionally, subjects with PWS having a Type 1 deletion sub-type have also been demonstrated to shown more insulin resistance leading to higher HbA1c levels at baseline. Since changes in serum magnesium are associated with changes in HbA1c, primarily in those PWS subjects with a measured baseline serum magnesium below 2.2 mg/dL, the population of subjects with PWS was narrowed from the whole population, to interrogate subjects with PWS having a Type I deletion subtype and being administered extended-release diazoxide choline for 1 year.

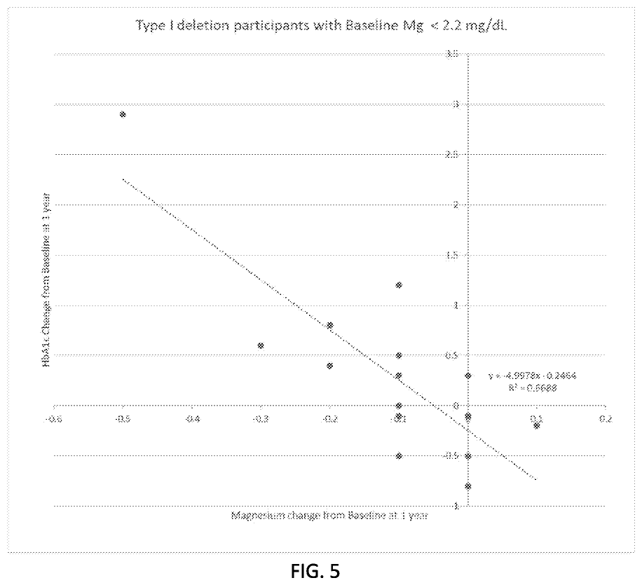

FIG. 5 depicts a graph of changes in measured serum magnesium levels and changes in measured HbA1c for subjects with PWS having a Type 1 deletion subtype, baseline serum magnesium levels less than 2.2 mg/dL and being administered extended-release diazoxide choline for 1 year compared to baseline levels. FIG. 5 indicates there is a strong negative relationship (e.g., negative slope of dotted line being about −4.9978) between change in serum magnesium and change in HbA1c in the Type 1 deletion subtype of subjects with PWS.

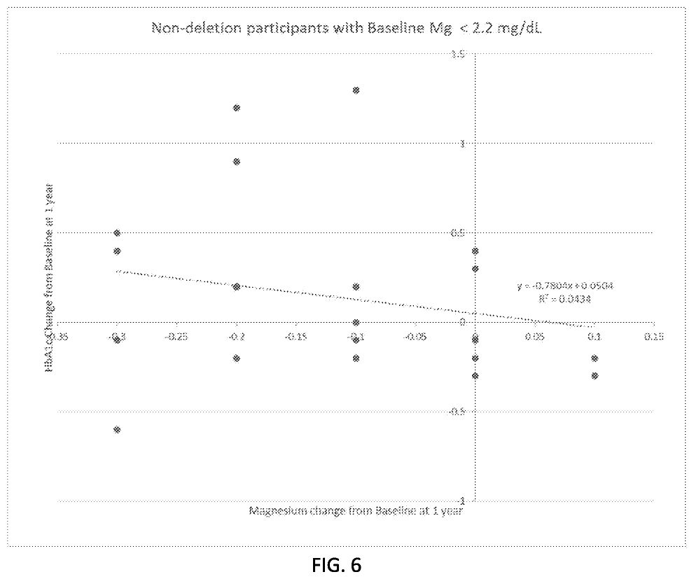

FIG. 6 depicts a graph of changes in measured serum magnesium levels and changes in measured HbA1c for subjects with PWS not having a Type 1 deletion subtype, baseline serum magnesium levels less than 2.2 mg/dL and being administered extended-release diazoxide choline for 1 year compared to baseline levels. As indicated in FIG. 6, there is strongly reduced negative relationship (e.g., negative slope of dotted line being about −0.7894) between change in serum magnesium and change in HbA1c in subjects with PWS lacking the Type 1 deletion subtype compared to subjects with PWS having the Type 1 deletion phenotype (having a negative slope of about −4.9978). The effect of changes in serum magnesium levels on HbA1c depends slightly on a genetic subtype of PWS (e.g., Type 1 deletion subtype vs non-Type 1 deletion subtype).

### Example 2.6 Obese Subjects with PWS Demonstrate a Negative Relationship Between Changes in Concentrations of Serum Magnesium and HbA1c after Administration of K_{ATP} Channel Opener

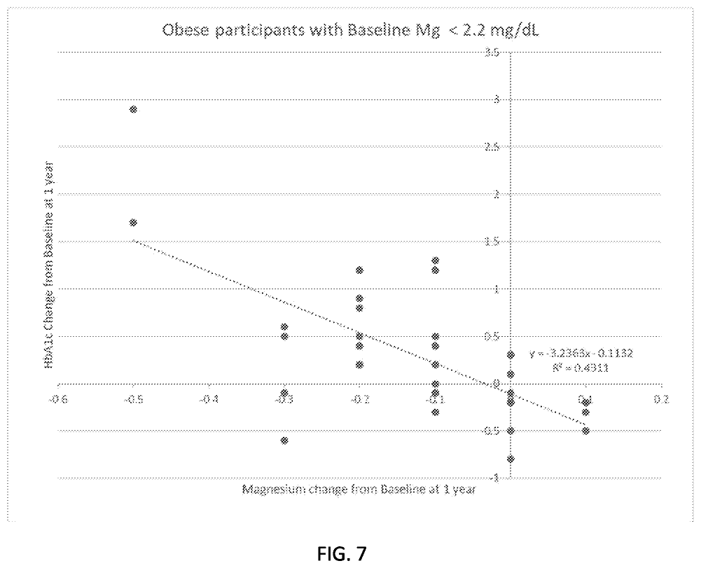

Due to hyperphagia in PWS, many subjects with PWS also demonstrate obesity. To interrogate this sub-population of subjects with PWS, the population of subjects with PWS was narrowed from the whole population to subjects with PWS administered extended-release diazoxide choline for 1 year, having a baseline measured serum magnesium level less than 2.0 mg/dL and being obese. Obesity was defined as an increase in body weight beyond the limitation of skeletal and physical requirement, as the result of an excessive accumulation of fat in the body (e.g., having a body mass index greater than 30 kg/m²). FIG. 7 depicts a graph of changes in measured serum magnesium levels and changes in measured HbA1c for subjects with PWS having a baseline serum magnesium levels less than 2.2 mg/dL, being administered extended-release diazoxide choline for 1 year compared to baseline levels and being obese. As indicated in FIG. 7, there is a negative relationship (e.g., negative slope of dotted line being about −3.2363) between change in serum magnesium and change in HbA1c in obese subjects with PWS. The effect of changes in serum magnesium levels on HbA1c depend slightly on obesity in subjects with PWS. Taken together, Examples 2.5-2.6 demonstrate that although the effect of changes in serum magnesium on HbA1c in subjects with PWS administered extended-release diazoxide choline for 1 year do depend on genetic subtype or obesity, the effect of these changes can mostly depend on baseline serum magnesium levels. This suggests that subjects with PWS with baseline serum magnesium levels less than 2.2 mg/dL may derive benefit from magnesium supplementation.

### Example 3 Examples of Co-Administrations of a K_{ATP} Channel Opener and a Magnesium Supplement on Changes in Concentration of HbA1c Levels in Subjects with PWS

### Example 3.1 Measurements in HbA1c after Co-Administration of a K_{ATP} Channel Opener and a Magnesium Supplement in Subjects with PWS

Subjects with PWS were identified and measurement of key endpoints (e.g., HbA1c, serum magnesium, and the like) to determine baseline measurements were performed. The subjects with PWS included subjects with PWS having serum magnesium levels greater than 2.2 mg/dL, between 2.0 mg/dL and 2.2 mg/dL, less than 2.0 mg/dL, having a Type 1 deletion, not having a Type 1 deletion, being obese, not being obese, and with an age of greater than 4 years of age.

The subjects with PWS were stratified into two groups: Group 1 may be administered a K_{ATP} channel opener for a first period of time and Group 2 may be co-administered a K_{ATP} channel opener (e.g., extended-release diazoxide choline) and a magnesium supplement for a first period of time (e.g., 12 or more months). During the first period of time, concentration of HbA1c and serum magnesium may be periodically measured and compared to baseline values of concentration of HbA1c and serum magnesium concentration.

Group 2 subjects co-administered extended-release diazoxide choline and the magnesium supplement may demonstrate either no change in HbA1c or an increase in HbA1c compared to baseline values and compared to Group 1 subjects administered K_{ATP} channel opener alone. HbA1c levels may be evaluated in PWS subjects at specific time points during the first period of time including at least 1 week, at least 4 weeks, at least 8 weeks, at least 13 weeks, at least 52 weeks, and 15 or more months. HbA1c may rise slightly or may demonstrate no change compared to baseline values including at least 0.1%, at least 0.2%, at least 0.3%, at least 0.4%, at least 0.5%, at least 0.6%, at least 0.7%, at least 0.8%, at least 0.9%, and at least 1.0% or more in subjects with PWS co-administered extended-release diazoxide choline and the magnesium supplement.

### Example 3.2 Changes in HbA1c after Co-Administration of a K_{ATP} Channel Opener and a Magnesium Supplement in Obese Subjects with PWS or Subjects with PWS Having a Type 1 Deletion

Group 2 subjects co-administered extended-release diazoxide choline and the magnesium supplement may be further stratified based on risk factors for hypomagnesemia including obesity or Type 1 deletion status. Group 2 obese subjects with PWS also may demonstrate a decrease or no change in concentration of HbA1c compared to baseline values and Group 1 alone. Group 2 subjects with PWS who may demonstrate Type 1 deletions also may demonstrate a decrease or no change in concentration of HbA1c compared to baseline values and compared to Group 2 subjects with PWS not having Type 1 deletions and Group 1 alone.

### Example 3.3 Changes in HbA1c after Co-Administration of a K_{ATP} Channel Opener and a Magnesium Supplement in Subjects with PWS Having Baseline Serum Concentrations of Less than 2.0 mg/dL or Between 2.0 mg/dL and 2.2 mg/dL

Group 2 subjects who may be co-administered extended-release diazoxide choline and the magnesium supplement may be further stratified based on baseline serum magnesium concentrations less than 2.0 mg/dL or between 2.0 mg/dL and 2.2 mg/dL. Subjects with PWS having a baseline serum magnesium concentrations less than 2.0 mg/dL may demonstrate a decrease or no change in HbA1c at all specific time points during the first time period compared to baseline values and Group 1 alone. Subjects with PWS having a baseline serum magnesium concentration between 2.0 mg/dL and 2.2 mg/dL may demonstrate a decrease or no change in HbA1c at all specific time points during the first time period compared to baseline values and Group 1 alone.

### Example 4 Examples of Co-Administration of a KAIP Channel Opener and a Magnesium Supplement on Changes in Concentration of Serum Magnesium in Subjects with PWS

### Example 4.1 Changes in Concentration of Serum Magnesium after Co-Administration of a K_{ATP} Channel Opener and a Magnesium Supplement in Subjects with PWS

Serum magnesium levels of Group 1 and Group 2 subjects may be compared. Group 2 subjects co-administered extended-release diazoxide choline and the magnesium supplement may demonstrate an increase in serum magnesium compared to baseline values and compared to Group 1 subjects administered K_{ATP} channel opener alone. Serum magnesium concentrations may be evaluated in subjects with PWS at specific time points during the first period of time including at least 1 week, at least 4 weeks, at least 8 weeks, at least 13 weeks, at least 52 weeks, and 15 or more months. Serum magnesium concentrations may rise slightly compared to baseline concentrations including greater than about 1.7 mg/dL, about 1.8 mg/dL, about 1.9 mg/dL, about 2.0 mg/dL, about 2.1 mg/dL, or about 2.2 mg/dL in Group 2 subjects who may have been co-administered extended-release diazoxide choline and the magnesium supplement.

### Example 4.2 Changes in Concentration of Serum Magnesium after Co-Administration of a K_{ATP} Channel Opener and a Magnesium Supplement in Subjects with PWS at Risk for or Demonstrating Hypomagnesemia

Group 2 subjects co-administered extended-release diazoxide choline and the magnesium supplement may be further stratified based on risk factors for hypomagnesemia including baseline serum magnesium levels less than 2.0 mg/dL, or between 2.0 mg/dL and 2.2 mg/dL, obesity status or Type 1 deletion status. Group 2 subjects having magnesium concentrations less than 2.0 mg/dL may demonstrate an increase in serum magnesium levels compared to baseline concentrations and Group 1 alone at each specific time point during the first period of time. Group 2 subjects having magnesium concentrations between 2.0 mg/dL and 2.2 mg/dL also may demonstrate an increase in serum magnesium concentrations compared to baseline values and Group 1 alone at each specific time point during the first period of time.

### Example 4.3 Changes in Concentration of Serum Magnesium after Co-Administration of a K_{ATP} Channel Opener and a Magnesium Supplement in Obese Subjects with PWS or Subjects with PWS having a Type 1 Deletion

Group 2 subjects co-administered extended-release diazoxide choline and the magnesium supplement may be further stratified based on risk factors for hypomagnesemia including obesity status or Type 1 deletion status. Group 2 obese subjects with PWS also may demonstrate an increase in serum magnesium levels compared to baseline values and Group 1 alone at each specific time point during the first period of time. Group 2 subjects with PWS with Type 1 deletions also may demonstrate an increase in serum magnesium levels compared to each of: baseline values, Group 2 subjects with PWS not having Type 1 deletions and Group 1 alone.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of managing glycemic control in a subject with Prader-Willi Syndrome in need thereof, comprising co-administering daily to the subject with Prader-Willi Syndrome in need thereof,
(i) a daily dose of a K_{ATP} channel opener, or a pharmaceutically acceptable salt thereof, and
(ii) a daily dose of a magnesium supplement,
for at least 1 week.

2.-29. (canceled)

30. An extended-release pharmaceutical composition, comprising:
8.80-26.40 weight % diazoxide choline;
30.00 weight % polyethylene oxide (WSR N750);
15.00 weight % polyethylene oxide (WSR 303); and
silicified microcrystalline cellulose.

31. The extended-release pharmaceutical composition of claim 30, comprising:
19.10 weight % silicified microcrystalline cellulose.

32. The extended-release pharmaceutical composition of claim 30, further comprising one or more excipients selected from:
a filler;
a flow aid;
a glidant; and
a lubricant.

33. The extended-release pharmaceutical composition of claim 30, further comprising one or more excipients selected from:
dibasic calcium phosphate dihydrate;
talc;
colloidal silicon dioxide; and
magnesium stearate.

34. The extended-release pharmaceutical composition of claim 30, comprising:
8.80-26.40 weight % diazoxide choline;
30.00 weight % polyethylene oxide (WSR N750);
15.00 weight % polyethylene oxide (WSR 303);
19.10 weight % silicified microcrystalline cellulose;
5.0-23.60 weight % dibasic calcium phosphate dihydrate;
1.33-2.00 weight % talc;
0.667-1.00 weight % colloidal silicon dioxide; and
1.5 weight % magnesium stearate, wherein the components total 100 weight %.

35. The extended-release pharmaceutical composition of claim 30, comprising:
8.80 weight % diazoxide choline;
30.00 weight % polyethylene oxide (WSR N750);
15.00 weight % polyethylene oxide (WSR 303); and
silicified microcrystalline cellulose.

36. The extended-release pharmaceutical composition of claim 35, comprising:
19.10 weight % silicified microcrystalline cellulose.

37. The extended-release pharmaceutical composition of claim 35, further comprising one or more excipients selected from:
a filler;
a flow aid;
a glidant; and
a lubricant.

38. The extended-release pharmaceutical composition of claim 35, further comprising one or more excipients selected from:
dibasic calcium phosphate dihydrate;
talc;
colloidal silicon dioxide; and
magnesium stearate.

39. The extended-release pharmaceutical composition of claim 35, comprising:
8.80 weight % diazoxide choline;
30.00 weight % polyethylene oxide (WSR N750);
15.00 weight % polyethylene oxide (WSR 303);
19.10 weight % silicified microcrystalline cellulose;
23.60 weight % dibasic calcium phosphate dihydrate;
1.33 weight % talc;
0.67 weight % colloidal silicon dioxide; and
1.5 weight % magnesium stearate.

40. The extended-release pharmaceutical composition of claim 1, comprising:
26.40 weight % diazoxide choline;
30.00 weight % polyethylene oxide (WSR N750);
15.00 weight % polyethylene oxide (WSR 303); and
silicified microcrystalline cellulose.

41. The extended-release pharmaceutical composition of claim 40, comprising:
19.10 weight % silicified microcrystalline cellulose.

42. The extended-release pharmaceutical composition of claim 40, further comprising one or more excipients selected from:
a filler;
a flow aid;
a glidant; and
a lubricant.

43. The extended-release pharmaceutical composition of claim 40, further comprising one or more excipients selected from:
dibasic calcium phosphate dihydrate;
talc;
colloidal silicon dioxide; and
magnesium stearate.

44. The extended-release pharmaceutical composition of claim 40, comprising:
26.40 weight % diazoxide choline;
30.00 weight % polyethylene oxide (WSR N750);
15.00 weight % polyethylene oxide (WSR 303);
19.10 weight % silicified microcrystalline cellulose;
5.0 weight % dibasic calcium phosphate dihydrate;
2.00 weight % talc;
1.00 weight % colloidal silicon dioxide; and
1.5 weight % magnesium stearate.

45. A method of treating hyperphagia in a subject having Prader-Willi Syndrome, comprising administering to the subject a therapeutically effective amount of the extended-release pharmaceutical composition of claim 30.

46. The method of claim 45, wherein the extended-release pharmaceutical composition is in the form of a tablet.

47. The method of claim 45, wherein the extended-release pharmaceutical composition is administered to the subject in an amount of about 25 mg to about 580 mg.

48. An extended-release pharmaceutical composition, comprising:
8.80-26.40 weight % diazoxide choline;
one or more sustained release agents; and
a combination of at least two fillers.

49. The extended-release pharmaceutical composition of claim 48, wherein the one or more sustained release agents comprises polyethylene oxide.

50. The extended-release pharmaceutical composition of claim 49, wherein the polyethylene oxide is present in the composition at a level of 45% by weight.

51. The extended-release pharmaceutical composition of claim 49, wherein the polyethylene oxide is a combination of polyethylene oxide (WSR N750) and polyethylene oxide (WSR 303).

52. The extended-release pharmaceutical composition of claim 49, wherein the polyethylene oxide is a combination of 30% by weight of polyethylene oxide (WSR N750) and 15% by weight of polyethylene oxide (WSR 303).

53. The extended-release pharmaceutical composition of claim 48, wherein at least one filler of the combination of at least two fillers is silicified microcrystalline cellulose.

54. The extended-release pharmaceutical composition of claim 53, comprising 19.10 weight % silicified microcrystalline cellulose.

55. The extended-release pharmaceutical composition of claim 48, comprising 8.80 weight % diazoxide choline or 26.40 weight % diazoxide choline.

56. The extended-release pharmaceutical composition of claim 48, further comprising one or more excipients selected from:
a filler;
a flow aid;
a glidant; and
a lubricant.

57. The extended-release pharmaceutical composition of claim 48, further comprising one or more excipients selected from:
dibasic calcium phosphate dihydrate;
talc;
colloidal silicon dioxide; and
magnesium stearate.

58. An extended-release pharmaceutical composition, comprising:
8.80-26.40 weight % diazoxide choline;
polyethylene oxide (WSR N750);
polyethylene oxide (WSR 303);
silicified microcrystalline cellulose;
dibasic calcium phosphate dihydrate;
talc;
colloidal silicon dioxide; and
magnesium stearate.
